(19) United States
(12) Patent Application Publication
(10) Pub. No.: US 2025/0189546 A1
(43) Pub. Date: Jun. 12, 2025

(54) METHODS FOR ANALYZING DRUG MOLECULES IN MAMMALIAN TISSUES

(71) Applicant: The Scripps Research Institute, La Jolla, CA (US)

(72) Inventors: Li YE, San Diego, CA (US); Benjamin F. CRAVATT, La Jolla, CA (US); Zhengyuan PANG, San Diego, CA (US)

(21) Appl. No.: 18/846,322

(22) PCT Filed: Mar. 15, 2023
(86) PCT No.: PCT/US23/64432

§ 371 Date: Sep. 12, 2024

Related U.S. Application Data
(60) Provisional application No. 63/321,294, filed on Mar. 18, 2022.

(51) Int. Cl. G01N 33/94; G01N 1/34

(52) U.S. Cl. CPC G01N 33/94; G01N 1/34

(57) ABSTRACT

The invention provides novel methods for analyzing (e.g., imaging) drugs in mammalian tissues, and methods for identifying drug targets and analyzing drug-target interactions.

### CROSS-REFERENCE TO RELATED APPLICATIONS

The subject patent application claims the benefit of priority to U.S. Provisional Patent Application No. 63/321,294 (filed Mar. 18, 2022; now pending). The full disclosure of the priority application is incorporated herein by reference in its entirety and for all purposes.

### STATEMENT OF GOVERNMENT SUPPORT

This invention was made with government support under grant numbers DK128800, DK114165 and DK124731 awarded by the National Institutes of Health. The government has certain rights in the invention.

### BACKGROUND OF THE INVENTION

Target identification and validation for small molecules has been a longstanding challenge in chemical biology and drug discovery. Although remarkable advances have been made to probe drug-target interactions at the molecular scale, these approaches are generally less suitable for such relationships at the tissue level in vivo. Traditional pharmacokinetics and pharmacodynamics often quantify drug concentrations or drug-target interactions in homogenized organs, eliminating the spatial resolution that is critical for understanding in vivo target engagement. Imaging-based methods, such as positron emission tomography (PET), are widely used to profile small molecule distribution in vivo, but lack sufficient resolution to differentiate drug binding states at the cellular level to precisely identify drug-target interactions. An ideal method should allow in situ visualization of target-bound drugs at single-cell resolution, while at the same time, being compatible with multiplexed molecular characterization of their drug-target interactions. These objectives are particularly important for drugs that target the central nervous system, which is markedly heterogeneous in cellular composition and spatial organization.

Fluorescence light microscopy has revolutionized high-resolution in situ imaging of endogenous biomolecules such as proteins and nucleic acids. However, exogenous small molecules are more difficult to image because appending a fluorescent tag alters the size and chemical properties of the parent compound, potentially distorting drug distribution and on- and off-target engagement. Biorthogonal reactions, including the Cu(I)-catalyzed azide-alkyne cycloaddition (CuAAC) click reaction, can partly address these problems by enabling incorporation of larger tags after a drug has reached its target using small and inert alkyne/azide handles. While this click chemistry (CC) strategy combined with chemical proteomic methods such as activity-based protein profiling (CC-ABPP) has proven effective for identifying drug targets in various biological settings, including animal models, such approaches have not yet enabled high-resolution spatial imaging of drug-target interactions in vivo due to the poor signal-to-noise ratio (SNR) in intact tissues.

There is a need in the art for more effective tools to observe drug-target interactions, especially at cellular resolution in intact tissues. The instant invention is aimed at addressing these and other unmet needs.

### SUMMARY OF THE INVENTION

In one aspect, the invention provides methods for analyzing an drug molecule or metabolite thereof in a mammalian tissue. These methods entail (a) obtaining a tissue sample from a mammalian subject who has been administered the drug molecule, (b) clearing the tissue sample with a delipidation agent, (c) performing a CuAAC reaction with the cleared tissue sample to label the drug molecule, and (d) examining the drug molecule in the tissue sample with an analytical means. In some methods, the drug molecule to be analyzed is a small molecule. In some methods, the drug molecule to be analyzed is a covalent drug. In different embodiments, the employed delipidation agent can be a hydrogel based tissue clearing agent, a hydrophobic tissue clearing agent, a hydrophilic tissue clearing agent, or a combination thereof.

In some preferred embodiments, the cleared tissue sample is subject to a CuAAC click reaction to label the drug molecule with a fluorophore. In some embodiments, the CuAAC reaction employed in the methods of the invention is optimized. In some of these embodiments, the optimized CuAAC reaction uses a Cu²⁺ concentration that is from about 100 μM to about 150 μM. In some embodiments, the CuAAC reaction is optimized by including a pre-incubation of the cleared tissue prior to initiation of the click reaction. In some embodiments, the CuAAC reaction is optimized with a click reaction ligand that improves signal to noise ratio. In various embodiments, the employed click reaction ligand can be, e.g., BTTP, THPTA or BTTAA.

Typically, the drug molecule to be analyzed in the methods of the invention, or a metabolite thereof, contains an alkyne group or an alkyne analog that suitable for a click reaction. In some preferred embodiments, the analytical means used to examine the drug molecule in the cleared and click reaction labeled tissue sample is an imaging means. For example, some preferred methods of the invention can employ confocal microscopy to examine the labeled drug molecule in the tissue sample.

In addition to examining the drug molecule in the cleared and click reaction labeled tissue sample, some methods of the invention can additionally include staining the tissue sample with an agent for a cell type that is known or suspected to express a target of the drug molecule. In some of these embodiments, the employed staining agent is an antibody or nucleic acid probe that is specific for the cell type.

In a related aspect, the invention provides methods for identifying the target of a drug molecule in a mammalian tissue. These methods involve (a) obtaining a tissue sample from a mammalian subject who has been administered the drug molecule, (b) clearing the tissue sample with a delipidation agent, (c) performing a CuAAC reaction with the cleared tissue sample to label the drug molecule, and (d) examining the drug molecule in the tissue sample with an analytical means to identify the target of the drug molecule in the tissue. Some of these methods are directed to identifying the targets of small molecule drug molecules. Some of these methods are directed to identifying the targets of covalent drug molecules. In various embodiments, the employed tissue clearing agent is a hydrogel based tissue clearing agent, a hydrophobic tissue clearing agent, or a hydrophilic tissue clearing agent. In some preferred embodiments, the CuAAC reaction for labeling the drug molecule is optimized. In various embodiments, the binding target in the tissue sample can be identified via, e.g., immunostaining, RNA hybridization, or a spatially-resolved molecular characterization means.

A further understanding of the nature and advantages of the present invention may be realized by reference to the remaining portions of the specification and claims.

### DESCRIPTION OF THE DRAWINGS

FIG. 1. Click reaction ligand optimization with CLARITY brain sections. TBTA is 90 μM. For all the other ligands, copper ligand ratio is 1:2. Reaction under CC-ABPP conditions (TBTA) generates high background in vehicle control. At 50 μM CuSO₄, only BTTP gives faint labeling. Increasing CuSO₄ concentration to 100 and 150 μM provides robust labeling without significant background labeling. At least 100 μM CuSO₄ is required. Unless otherwise noted, 150 μM CuSO₄, 300 μM BTTP was used in all CATCH studies. Images represent primary somatosensory cortex (S1). Scale bar, 20 μm.

FIG. 2. CATCH labeling in PFA fixed and hydrogel-based clearing techniques (CLARITY and SHIELD) cleared tissues. Images represent S1. Scale bar, 20 μm.

FIG. 3. For efficient and homogeneous drug labeling, CATCH requires tissue samples to be pre-incubated in click incubation buffer (5 μM Alexa647-picolyl-azide, 150 μM CuSO₄, 300 μM BTTP, 10% DMSO in PBS) overnight prior to reaction initiation. Tissue samples are then transferred to new incubation buffer. Sodium ascorbate (NaAsb) is added to the incubation buffer to obtain the click reaction buffer (5 μM Alexa647-picolyl-azide, 150 μM CuSO₄, 300 μM BTTP, 2.5 mM NaAsb, 10% DMSO in PBS). Images represent S1. Scale bar, 20 μm.

FIG. 4. CATCH is compatible with drug imaging in liver and small intestine. Scale bar, 50 μm.

FIG. 5. CATCH is compatible with molecular marker staining for cell type identifications. Figure shows PF7845-yne in S1 binding NeuN positive neurons, but not lectin positive blood vessels. Images represent S1. Scale bar, 10 μm.

FIG. 6. CATCH is compatible with reversible molecular marker staining. Drug-fluorophore complex is resistant to antibody elution (A). After primary antibody staining, NeuN staining can be removed without compromising click labelled pargyline-yne positive cells. Tissues is re-stained with TH antibody for reversible, multiplexed cell type identifications (B). Images represent pons. Scale bar, 10 μm.

FIG. 7. CATCH can resolve drug binding with subcellular resolution. Figure shows MJN110-yne binding in presynaptic terminals marked by synpasin staining, indicating MJN110-yne binding to monoacylglycerol lipase (MAGL). Figure represents hippocampus CA1. Scale bar, 10 μm.

FIG. 8. CATCH can enable spatially resolved high resolution pharmacology. Schematics for dose-dependent in vivo blocking studies. At low dose, parental drug does not fully block target, leaving space for subsequent alkyne drug binding. At high dose, parental drug saturates target, thereby abolishing alkyne drug binding (A). Zoomed-out views of PF7845 blocking at indicated dose followed by PF7845-yne injection (1 mg/kg, 1 h, i.p.) in hippocampus and cortex. Cortical PF7845-yne is fully blocked with 0.02 mg/kg PF7845. But residue unoccupied FAAH is still present in hippocampus. Scale bar, 500 μm (B). Schematics for dose-dependent direct alkyne drug mapping. Higher target saturation should associate with higher CATCH labeling intensity (C). Zoomed-out view of PF7845-yne binding dynamics in hippocampus and cortex (4 h, i.p.). PF7845-yne saturates cortical, but not hippocampal FAAH at 0.1 mg/kg. Scale bar, 500 μm (D).

FIG. 9. CATCH can resolve potential drug off target binding regions. PF7845is considered a safe FAAH inhibitor whereas BIA 10-2474 is a FAAH inhibitor associated with clinical neurological toxicities. 5 m/kg dosing was used for both drugs to maximize the contrast of target visualization. Full coronal sections at Bregma −4.4 with zoomed-in views at periaqueductal gray (PAG) and reticulo tegmental nucleus of the pons (RtTg). BIA10-2474-yne (5 mg/kg, 4 h, i.p.) binds to large cellular structures in wildtype, but not FAAH−/− mice PAG, suggesting primarily on target binding in PAG. Cellular structures can be found in RtTg in FAAH−/− mice treated with BIA10-2474-yne, but not with PF7845-yne, indicating off target binding. Image brightness/contrast is optimized for each brain region for better visualization. Scale bar, 100 μm.

### DETAILED DESCRIPTION

### I. Overview

Comprehensive understanding in vivo efficacy and toxicity with high spatial and resolution remains a major unmet challenge for realizing the full potential of chemical probes and drugs. Currently, it is impossible to determine drug binding across a whole animal (multiple organ-systems) with cellular and molecular resolution in mammals. Biochemical methods like chemoproteomics provide excellent molecular details of drug-target interactions but do not have the spatial resolution to identify tissue and cellular targets. Currently imaging methods like PET resolve greater tissue distribution but fall short of assessing molecular and cellular specificity.

The invention is predicated in part on the studies undertaken by the inventors to develop a method to optically image covalent drug targets in intact mammalian tissues. The method, termed Clearing Assisted Tissue Click Chemistry (CATCH), permits specific and robust in situ fluorescence imaging of target-bound drug molecules at subcellular resolution and enables the identification of target cell types. As exemplified herein, using well-established inhibitors of endocannabinoid hydrolases and monoamine oxidases, direct or competitive CATCH not only reveals distinct anatomical distributions and predominant cell targets of different drug compounds in the mouse brain, but also uncovers unexpected differences in drug engagement across and within brain regions, reflecting rare cell types, as well as dose-dependent target shifts across tissue, cellular and subcellular compartments that are not accessible by conventional methods. CATCH represents a valuable and differentiated platform for interrogating in vivo small molecule-target interactions.

The invention accordingly provides novel methods for analyzing target-bound drug molecules and related methods for identifying drug targets in tissues. Detailed steps and protocols for performing these methods are described in detail herein. As demonstrated herein, methods of the invention can be used to analyze (e.g., to image) drugs at sub-cellular resolution in native tissue. The binding targets can be subsequently identified using immunostaining, RNA hybridization, or other spatially-resolved molecular characterization approaches. In vivo tissue distribution and target identities of the drug could be ascertained from the same platform to obtain the comprehensive on-and off-target map of candidate drugs.

The invention can employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Sambrook et al, ed. (1989) Molecular Cloning A Laboratory Manual (2nd ed.; Cold Spring Harbor Laboratory Press); Sambrook et al, ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al, eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); and in Ausubel et al. (1989) Current Protocols in Molecular Biology (John Wiley and Sons, Baltimore, Md.).

### II. Definitions

The following sections provide more detailed guidance for making and using the compositions of the invention, and for carrying out the methods of the invention. Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

It is noted that, as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “alkyne-based compound” refers to a small molecule drug or a polymeric molecule comprising at least one reactive alkynyl group. An “alkynyl group” is an unsaturated, linear or branched or cyclic hydrocarbon group consisting at least one carbon-carbon triple bond. In one embodiment, the alkyne-based substrate comprises preferably at least one terminal alkynyl group (—C≡CH).

As used herein, the term “azide-based substrate” refers to a small molecule or a polymeric molecule comprising at least one azide group. The substrate contemplated within the invention may comprise a soluble reagent or a solid-immobilized reagent, such as a surface-immobilized reagent.

The term “specific binding” or “specifically binds to” or is “specific for” refers to the binding of a binding moiety to a binding target, such as the binding of an immunoglobulin to a target antigen, e.g., an epitope on a particular polypeptide, peptide, or other target (e.g. a glycoprotein target), and means binding that is measurably different from a non-specific interaction (e.g., a non-specific interaction can be binding to bovine serum albumin or casein). Specific binding can be measured, for example, by determining binding of a binding moiety, or an immunoglobulin, to a target molecule compared to binding to a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a K_{d} for the target of at least about 200 nM, alternatively at least about 150 nM, alternatively at least about 100 nM, alternatively at least about 60 nM, alternatively at least about 50 nM, alternatively at least about 40 nM, alternatively at least about 30 nM, alternatively at least about 20 nM, alternatively at least about 10 nM, alternatively at least about 8 nM, alternatively at least about 6 nM, alternatively at least about 4 nM, alternatively at least about 2 nM, alternatively at least about 1 nM, or greater. In certain instances, the term “specific binding” refers to binding where a molecule binds to a particular polypeptide or epitope on a particular polypeptide without substantially binding to any other polypeptide or polypeptide epitope.

The terms “conjugate,” “conjugated,” and “conjugation” refer to any and all forms of covalent or non-covalent linkage, and include, without limitation, direct genetic or chemical fusion, coupling through a linker or a cross-linking agent, and non-covalent association.

The term “tissue clearing” as used herein refers to a process that has the effect of tuning, matching, or homogenizing the refractive index (RI) of tissue, generally resulting in an increase in the transparency of the tissue. The transparency of the tissue can be quantitatively determined via optical absorption spectrophotometry, such as measuring light transmission through the tissue, or confocal microscopy.

The term “refractive index” or “RI” as used herein refers to the ratio of the speed of radiation (such as in electromagnetic radiation or light) in one medium (such as air, glass, or a vacuum), to that in another medium.

The term “derivative” refers to chemical compounds/moieties with a structure similar to that of a parent compound/moiety but different from it in respect to one or more components, functional groups, atoms, etc. The derivatives can be formed from the parent compound/moiety by chemical reaction(s). The differences between the derivatives and the parent compound/moiety can include, but are not limited to, replacement of one or more functional groups with one or more different functional groups or introducing or removing one or more substituents of the hydrogen atoms. In some forms, the derivatives can also differ from the parent compound/moiety with respect to the protonation state. In some forms, the derivatives can be derived from the parent compound/moiety via an acid-base reaction. Preferably, the derivatives retain the bioactivity of the parent compound/moiety, such as at least 100%, 95%, 90%, 85%, 80%, 75%, 70%, 65%, and 60% of the bioactivity of the parent compound/moiety. In some forms, the derivatives possess higher activity compared to the parent compound/moiety.

The term “target” or “binding target” is used in the broadest sense and specifically includes polypeptides, without limitation, nucleic acids, carbohydrates, lipids, cells, and other molecules with or without biological function as they exist in nature. In some preferred embodiments herein, the target is a cell surface antigen or a receptor.

The term “antigen” refers to an entity or fragment thereof, which can bind to an immunoglobulin or trigger a cellular immune response. An immunogen refers to an antigen, which can elicit an immune response in an organism, particularly an animal, more particularly a mammal including a human. The term antigen includes regions known as antigenic determinants or epitopes, as defined above.

### III. Tissue Clearing and Delipidation

The invention provides methods for analyzing drug molecules (e.g., in situ imaging) and for detecting drug targets in mammalian tissues at cellular resolution. The drug molecules suitable for analysis of the methods of the invention can be of any chemical nature. These include, e.g., small molecule compounds, peptides, proteins, antibodies, nucleic acids, lipids, polysaccharides, or any combinations thereof. In some embodiments, methods of the invention are directed to analysis of small molecule drugs. In some preferred embodiments, the drug molecule is an alkyne-based compound, which contains an alkyne group or analog thereof that is suitable for click chemistry reactions. In some preferred embodiments, the drug molecule to be analyzed with methods of the invention are covalent drugs. Covalent drugs block protein function by forming a specific bond between the ligand and target protein. A covalent mechanism of action can provide many pharmacological advantages over a reversible mechanism of action; these advantages include enhanced potency, selectivity, and prolonged duration of action.

In accordance with methods of the invention, a tissue sample is first obtained from a mammalian subject who has been administered a drug molecule. The tissue sample is then cleared with a delipidation agent. The cleared tissue sample is subject to a CuAAC reaction thereafter to label the drug molecule. Further analyses, e.g., imaging, can be performed to analyze the drug molecule in situ (e.g., its spatial distribution) and/or to identify targets of the drug molecule at cellular or subcellular resolution.

Delipidation of a tissue sample isolated from a subject can be performed with various techniques that are well known and routinely practiced in the art. In general, these include hydrogel-based tissue clearing, hydrophobic-based tissue clearing, hydrophilic tissue clearing, and other hybrid tissue clearing methods combining, for example, hydrogel-and hydrophobic-based tissue clearing. In some embodiments, delipidation to clear the tissue can be performed with the methods specifically demonstrated herein, e.g., HYBRID, CLARITY, and SHIELD. Hydrogel based tissue clearing involves embedding tissue samples on a hydrogel matrix to create a cross-linked tissue/gel hybrid containing the fixed proteins and RNA. These methods use ionic detergents for delipidation, which can be enhanced by electrophoresis without causing significant loss of biomolecules. See, e.g., Chung et al., Nature 497, 332-337, 2013; Murray et al., Cell 163, 1500-1514, 2015; Ku et al., Nat. Biotechnol. 34, 973-981, 2016; Woo et al., Exp. Mol. Med. 48, e274, 2016; Woo et al., Biochem. Biophys. Res. Commun. 524, 346-353, 2020; Woo et al., Int. J. Mol. Sci. 22,2892, 2021; Gradinaru et al., Annu. Rev. Biophys. 47, 355-376, 2018; and Choi et al., Cell 184, 4115-4136, 2021. Hydrogel-based methods have the advantages of preserving endogenous FP signal, retaining a higher proportion of biomolecules, and better preserving brain anatomy and ultrastructure. On the downside, it usually involves longer and more complex protocols (compared with hydrophobic-and hydrophilic-methods) in requiring in situ hydrogel polymerization. It may also require active (electrophoretic) labeling and clearing.

Methods of the invention can also utilize hydrophobic (organic solvent-based) clearing protocols. These methods rely on tissue dehydration, delipidation and permeabilization using organic solvents such as ethanol, methanol, tetrahydrofuran (THF), and tert-butanol. See, e.g., Dodt et al., Nat. Methods 4, 331-336, 2007; Renier et al., Cell 159, 896-910, 2014; Erturk et al., Nat. Protoc. 7, 1983-1995, 2012; and Pan et al., Nat. Methods 13, 859-867, 2016. Most protocols known for hydrophobic clearing involve simple tissue immersion (passive clearing) in a graded series of increasing solvent concentration. However, active clearing protocols in which the solvents are perfused through the circulatory system allow clearing entire rodent bodies and large organs such as the human brain. Further incubation in dichloromethane (DCM) may improve tissue delipidation in large samples, at the expense of decreasing fluorescence from fluorescent proteins (FP). After dehydration, samples are immersed in a clearing solution consisting of a mixture of benzyl alcohol and benzyl benzoate (BABB) or dibenzyl ether (DBE), to achieve RI homogenization and thus transparency. Hydrophobic-based tissue clearing provides very quick and reproducible protocols requiring simple immersion. It has higher clearing performance (transparency). However, endogenous FP signal is lost in hydrophobic-based tissue clearing. There is a complete loss of lipids and associated biomolecules, and also possible loss of proteins and nuclei acids. Further, hydrophobic-based tissue clearing may require solvent-resistant materials and optics, and involves the use of volatile and toxic solvents.

Tissue clearing in the methods of the invention may also utilize hydrophilic reagents. Examples of suitable hydrophilic reagents include different detergents (such as Triton-X100, saponin, sodium dodecyl sulfate (SDS), and etc.) for permeabilization and delipidation, and high-refractive index aqueous solutions containing sugars (fructose, sucrose, sorbitol), urea, aminoalcohols, or different combinations of the former. See, e.g., Ke et al., Nat. Neurosci. 16, 1154-1161, 2013; Hama et al., Nat. Neurosci. 14, 1481-1488, 2011; Susaki et al., Cell 157, 726-739, 2014; Kuwajima et al., Development 140, 1364-1368, 2013; Yu et al., Sci. Rep. 8, 1964, 2018; and Zhu et al., Proc. Natl. Acad. Sci. USA 116, 11480-11489, 2019. Hydrophilic tissue clearing protocols have the advantages are preservation of endogenous FP signal, use of safer reagents and compatibility with standard materials and optics. The drawback is that hydrophilic tissue clearing may require longer incubation times, that protocols using high detergent concentrations may cause loss of lipids and associated biomolecules, proteins and nuclei acids.

In some embodiments of the invention, delipidation to render tissues transparent can be performed with the specific tissue clearing protocols exemplified herein. For example, delipidation of some tissue samples can be performed with CLARITY (clear lipid-exchanged acrylamide-hybridized rigid imaging-compatible tissue-hydrogel), a hydrogel-based tissue clearing technique exemplified herein (see, e.g., Chung et al., Nature 497, 332-337,2013). CLARITY transforms intact tissue into a nanoporous hydrogel-hybridized form (crosslinked to a three-dimensional network of hydrophilic polymers) that is fully assembled but optically transparent and macromolecule-permeable. CLARITY enables intact-tissue in situ hybridization, immunohistochemistry and antibody labelling. This allows fine structural analysis of clinical samples, in a form suitable for probing the underpinnings of physiological function and disease. Using CLARITY, fixed tissues (e.g., brain tissue samples) are subject to incubation and agitation with an A1P4 hydrogel, as specifically exemplified herein. This is followed by degassing and washing before labeling reactions. In some embodiments, another hydrogel-based tissue clearing method described herein, SHIELD (stabilization under harsh conditions via intramolecular epoxide linkages to prevent degradation), can be employed. This delipidation method uses epoxide monomer to form controlled intra-and intermolecular cross-link with biomolecules. SHIELD preserves protein fluorescence and antigenicity, transcripts and tissue architecture under a wide range of harsh conditions. See, e.g., Park et al., Nat. Biotechnol. 37, 73-83, 2019. In some other embodiments, hydrophobic tissues can be cleared via fDISCO and iDISCO exemplified herein, which uses THF and methanol to remove lipid component. More detailed guidance of these two tissue clearing protocols is provided in the published literature, e.g., Renier et al., Cell 159, 896-910, 2014; Qi et al., Sci. Adv. 5, eaau8355, 2019; and Erturk et al., Nat. Protoc. 7, 1983-1995, 2012. In still some other embodiments, clearing of hydrophilic tissues can be carried out with CUBIC3.0 to remove lipid with water soluble detergent. This tissue clearing method can be performed as described herein or in the published literature (e.g., Tainaka et al., Cell Rep 24, 2196-2210, 2018).

### IV. Labeling Drug Molecules in Cleared Tissue Samples via Click Reactions

Once the tissue sample has been cleared and delipidated with any of the protocols described above, it is then subject to labeling of the drug molecule or metabolite thereof that is present in the tissue sample. The labeling is achieved by attaching or affixing a detectable label (e.g., a fluorescent agent) to the drug molecule or metabolite thereof. In some preferred embodiments, the drug molecule or metabolite thereof is labeled with a fluorophore via click chemistry reactions (or click reactions). Click Chemistry is defined as any chemical reaction that allows high yields, generates no side-products or ones that are easily removed, is stereospecific, gives physiologically stable products, exhibits a large thermodynamic driving force, and has simple reaction conditions. See, e.g., Kolb et al., Angew. Chem., Int. Ed. 2001, 40, 2004-2021; Kolb et al., Drug Discovery Today 2003, 8, 1128-1137. Unless otherwise noted, the click reaction used in the practice of the invention is the CuAAC reaction. CuAAC click reaction is well known and routinely practiced in the art. It is a type of Huisgen1,3-dipolar cycloaddition based on the formation of 1,4-disubstituted [1,2,3]-triazoles between a terminal alkyne and an aliphatic azide in the presence of copper. The reaction has found increasing applications in all aspects of drug discovery in medicinal chemistry, such as for generating lead compounds through combinatorial methods. Bioconjugation via click chemistry is rigorously employed in proteomics and nucleic research. Click reaction is used to label and detect a molecule in samples that would be compromised by direct labeling or antibody-based secondary detection techniques. The click label is small enough to penetrate complex samples easily, and the selectivity and stability of the click reaction provide high sensitivity and low background signal. Labeling a cleared tissue sample in the practice of the methods of the invention can be readily carried out in accordance with the protocols that are well known and routinely practiced in the art.

In some embodiments, the click reaction used in the methods of the invention for labeling the drug molecule or metabolite in the cleared tissue samples is an optimized or modified CuAAC reaction that provides improved imaging quality. In some embodiments, the modified click reaction uses unconventional CuAAC reaction conditions that enable sufficient signal to noise ratio (SNR) while maintaining low background signal in vehicle controls. In some embodiments, the modified click reaction employs newer generation click reaction ligands or azide-based substrates. Examples of such newer click reaction ligands include, e.g., THPTA, BTTAA and BTTP, as exemplified herein. In some embodiments, the modified click reaction uses a Cu concentration not commonly used in conventional CuAAC reactions. As exemplified herein, some methods of the invention can use a Cu concentration in the click reaction that is between about 100 μM and about 300 μM. In some embodiments, a Cu concentration of from about 100 μM to about 150 μM can be used. In one preferred embodiment, the employed click reaction ligand is BTTP, and a Cu concentration of 150 μM is used as exemplified herein.

In some embodiments, the optimized click reaction involves a pre-incubation step of the cleared tissue sample prior to initiation of the click reaction. In this step, the cleared tissue sample is pre-incubated in an incubation buffer for a period of time that is sufficient to condition the tissue for homogenous labeling across the tissue. In various embodiments, the pre-incubation period can last at least 1 hour, 2 hours, 4 hour, 6 hours, 8 hours, 10 hours, 12 hours, 14 hours or longer. In some embodiments, the incubation buffer can contain Alexa647-picolyl-azide, CuSO4, BTTP and DMSO in appropriate concentrations detailed herein. After the pre-incubation, the treated tissue sample is then transferred to click reaction buffer to initiate the click reaction. As exemplified herein, the click reaction buffer can contain fresh incubation buffer with the addition of sodium ascorbate (NaAsb).

As a specific exemplified embodiment of optimized click reaction, the incubation buffer can contain 5 μM Alexa647-picolyl-azide, 150 μM CuSO₄, 300 μM BTTP, 10% DMSO in PBS. The pre-incubation period can last about 1-16 hours. The full click reaction buffer contains 5 μM AF647-picolyl azide, 150 μM CuSO_{4˜,} 300 μM BTTP, 2.5 mM sodium ascorbate, and 10% DMSO in PBS. Detailed protocols for carrying out the optimized click reactions are exemplified herein.

### V. Analysis of Cleared and Labeled Tissues

To ascertain presence of the drug molecule and/or to identify its target in the tissue obtained from a subjected administered with the drug, the cleared and labeled tissue is then analyzed with an appropriate means. Depending on the tissue sample and the detectable label used in the click reaction, various physical, optical, immunochemical or biological approaches may be used. These include, e.g., confocal microscopy, fluorescent imaging, mass spectrometry imaging, multiphoton microscopy, fluorescence light microscopy, and positron emission tomography (PET). In various embodiments, analysis of drugs in situ in biological samples can be readily performed using these and other tools that have been routinely practiced in the art. See, e.g., Diaspro et al., Microsc. Res. Tech. 1999; 47:163-4; Denk et al., Science1990; 248:73-6; Imbert et al., Methods Mol. Biol. 1999; 122:341-55; Tadrous et al., J. Pathol. 2000; 191:345-54; Xue et al., Sci. Adv. 2018; 4: eaat9039; McMahon et al., Proc. SPIE Int. Soc. Opt. Eng. 2020 February; 11219; and Bian et al., STAR Protocols 2022; 3:101600. In some preferred embodiments, spatial distribution and target interaction of the drug molecule in the cleared tissue can be examined first via an imaging means. In addition to confocal microscopy exemplified herein, other suitable imaging means may also be employed in the practice of the invention, e.g., fluorescence lifetime imaging, Raman imaging, and autoradiography.

As noted above, the drug molecule or metabolite is labeled with a detectable label such as a fluorophore via the CuAAC reaction. The cleared and labeled tissue sample can then be examined via a suitable means to analyze the labeled drug molecule in the tissue. In some preferred embodiments, presence of the drug molecule and/or its interaction with its target in the tissue are analyzed with confocal microscopy as exemplified herein. Confocal microscopy is based on the illumination of the biological specimen (e.g., a tissue) by an excitation light followed by the detection of the emitted light. This implies that only fluorescent objects can be examined. In most cases, the biological specimen is made fluorescent by the use of labels directed to specific intracellular or intratissular structures (confocal epifluorescence). Over the years, the spontaneous autofluorescence of many biological molecules (confocal epitransmission) or the capacity of opaque surfaces to reflect light (confocal reflectance) have been exploited.

In addition to imaging, the cleared and click reaction labeled tissue sample can be further subject to cell type registration. In some of these embodiments, the tissue sample can be stained with an agent for a cell type that is known or suspected to express a target of the drug molecule. Cells expressing a specific target molecule or marker of interest may be identified via various assays. These include, e.g., flow cytometry, immunohistochemistry, and next generation sequencing. In some embodiments, the tissue sample can be stained with antibody or nucleic acid probes that are specific for one or more marker molecules of the cell type.

Cellular markers of a great number of cell types including diseased cells (e.g., tumor cells) are well known and extensively characterized in the art. Using human immune cells for illustration, some well characterized cell markers include, e.g., CD34+, CD38−, CD45RA−, CD49+, CD90/Thy 1+for hematopoietic stem cells (HSC); CD34+, CD38−, CD45RA−, CD90/Thy 1− for multi-potent progenitor cells (MPP); CD34+, CD38+, CD10+, CD45RA+ for common lymphoid progenitor (CLP); CD34+, CD38+, CD7−, CD10−, CD45RA−, CD90/Thyl−, CD135+ for common lymphoid progenitor (CLP); CD34+, CD38+, CD7−, CD10−, CD45RA−, CD135−, IL3Rα− for megakaryocyte-erythroid progenitor (MEP); CD34+, CD38+, CD10−, CD45RA+, CD123+, CD135+ for granulocyte-monocyte progenitor (GMP); CD3−, CD56+, CD94+, NKp46+ for nature killer cells; CD3+for T-cells; CD19+ for B-cells; CD14+ for monocytes; CD11b+, CD68+, CD163+ for macrophage; CD11c+ and HLA-DR+ for dendritic cells; CD11b+, CD16+, CD18+, CD32+, CD44+, CD55+ for neutrophil; CD45+, CD125+, CD193+, F4/80+, Siglec-8+ for eosinophil; CD19−, CD22+, CD45low, CD123+ for basophil; CD32+, CD33+, CD117+, CD203c+, FcεRI+ for mast cell; CD235a+ for erythrocyte; CD41b+, CD42a+, CD42b+, CD61+for megakaryocyte; and CD41+, CD42a+, CD42b+, CD61+ for platelet. For any of these cell types, any one or combination of these known markers can be probed in order to ascertain the cell type in the tissue that is targeted by the drug molecule,

In some embodiments, the cellular maker to be detected from the tissue sample is specific to a deceased cell. In some of these embodiments, the cell marker is a tumor specific marker. Examples of such markers include, but are not limited to, HER2 (ERBB2), prostate-specific antigen (PSA), Prostatic acid phosphatase (PAP), CA 125, carcinoembryonic antigen (CEA), alpha-fetoprotein (AFP), human chorionic gonadotropin (HCG), CA 19-9, CA 15-3, CA 27-29, lactate dehydrogenase (LDH), and neuron-specific enolase (NSE).

### EXAMPLES

The following examples are provided to further illustrate the invention but not to limit its scope.

### Example 1. CATCH Enables Specific Labeling and Imaging of Drugs in Situ

Over the past decade, there has been a resurgent interest in chemical probes and drugs that operate by a covalent mechanism, as their irreversible engagement of protein targets can improve potency of engagement for challenging targets and afford superior pharmacodynamic properties in vivo. The irreversibility also makes it particularly crucial to understand the cellular targets of covalent drugs with high precision to minimize toxicity. To establish a model system from which the visualization of drug targets could be evaluated, the inventors explored a set of well-characterized covalent inhibitors of endocannabinoid hydrolytic enzymes, with the potential to treat a range of human neurological disorders. Initial experiments were performed using the fatty acid amide hydrolase (FAAH) inhibitor PF-04457845 (or PF7845), whose urea backbone forms a covalent bond with the active serine residue of FAAH to irreversibly inhibit enzyme activity. Previous work has shown that an alkyne-modified analogue PF7845-yne retained similar selectivity as PF7845 and was able to selectively detect FAAH in whole-brain lysates by CC-ABPP combined with an in-gel fluorescence readout (SDS-PAGE) (Niphakis et al., ACS Chem Neurosci 3, 418-426, 2012). However, attempts to directly image drug-bound FAAH in brain sections from mice treated with PF7845-yne with an azide-Alexa647 tag under CC-ABPP condition failed to reveal the cellular target due to poor SNR.

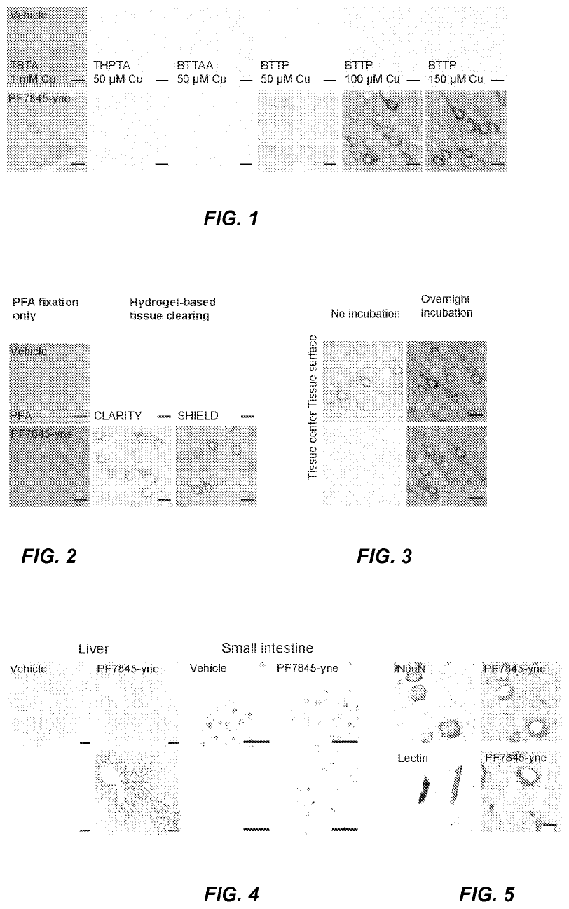

The inventors reasoned that the complex composition of tissue, particularly dense lipid membranes, might hinder the CuAAC reaction. Therefore, the inventors tested whether CLARITY, a polyacrylamide-based aqueous clearing method, could improve the SNR of CuAAC labeling. After applying CC-ABPP click conditions to brain sections from mice treated with PF7845-yne, CLARITY treatment rendered a membrane-like fluorescence signal in the primary somatosensory cortex (S1) after clicking with an Alexa-647-picolyl-azide, consistent with the expected FAAH engagement. However, background nuclear signal was observed in vehicle-treated samples, indicating the occurrence of substantial side reactions under this condition (FIG. 1).

Because a ligand is used to protect the catalytic Cu (I) from generating excessive reactive oxygen species (ROS) and side reactions with CuAAC, the inventors speculated that although the original CC-ABPP protocol using 1 mM CuSO₄ and the first-generation ligand TBTA yields satisfactory results in homogenized systems, the high concentration of Cu²⁺ may lead to significant side reactions in intact tissues. The inventors thus compared a range of newer generation ligands that allow lower concentration of Cu²⁺. THPTA, BTTAA and BTTP (Li and Zhang, 2016) significantly eliminated the background signal in vehicle-treated samples. Furthermore, BTTP resulted in weak but distinct membrane-like signals in PF7845-yne treated tissue using only 50 μM CuSO₄ (FIG. 1). By gradually increasing Cu²⁺ concentration, the inventors found that 150 μM CuSO₄ resulted in robust fluorescent labeling with minimal background staining in controls (FIG. 1). As expected, fluorescent labeling was strictly dependent on the presence of each component of the CuAAC reaction. Removing any of the reaction component (CuSO₄, BTTP, Alexa-647-picolyl azide) would disable click labeling. Importantly, rendering tissues optically transparent using reflective index matching alone was not sufficient to generate a satisfactory SNR, suggesting that the delipidation associated with tissue clearing was necessary to facilitate in situ CuAAC labeling. The inventors found after tissue clearing, directly labeling samples in full click reaction cocktail (5 μM Alexa647-picolyl-azide, 150 μM CuSO₄, 300 μM BTTP, 2.5 mM NaAsb, 10% DMSO in PBS) only provides faint labeling on the tissue surface (FIG. 3). Insufficient click labeling depth can be resolved by preincubating samples in click incubation buffer (5 μM Alexa647-picolyl-azide, 150 μM CuSO₄, 300 μM BTTP, 10% DMSO in PBS). Without NaAsb, Cu-BTTP complex can diffuse into tissue prior to initiating reaction, thereby ensuring homogenous catalytic species distribution across whole tissue depth (FIG. 3). After tissue clearing, similar results were achieved with different drug delivery routes including oral, intraperitoneal, and subcutaneous. The inventors demonstrated CATCH can be further applied to non-brain peripheral tissues (FIG. 4). In addition to liver and small intestine, the inventors have demonstrated drug mapping in large intestine, heart, spleen, colon, lung, kidney, and skeleton muscle. The inventors demonstrated CATCH is compatible with CLARITY and SHIELD (Park et al., Nat Biotechnol 37, 73-83, 2019), two hydrogel-based tissue clearing techniques (FIG. 2). In addition, the inventors demonstrated tissues cleared by hydrophobic tissue clearing methods like DISCOs (Erturk et al., Nat Protoc 7, 1983-1995, 2012; Qi et al., Sci. Adv. 5, eaau8355, 2019; Renier et al., Cell 159, 896-910, 2014), hydrophilic tissue clearing techniques like CUBIC3.0 (Tainaka et al., Cell Rep 24, 2196-2210, 2018), and other hybrid tissue clearing techniques like HYBRID (Nudell et al., Nat. Methods 19, 479-485, 2022) all yielded similar labeling profiles after in situ click reactions (FIG. 2). For simplicity, the inventors chose to adopt a combination of CLARITY, 150 μM CuSO₄ and 300 μM BTTP as the standard CATCH protocol for all subsequent experiments.

The inventors next sought to determine whether CATCH signals were specific to in vivo PF7845-yne engagement of FAAH by first performing a pharmacological blocking experiment. Pre-treatment with the parent compound PF7845 completely abolished PF7845-yne-generated CATCH signals across the brain. The inventors also compared PF7845-yne CATCH signals in wild-type (FAAH+/+) mice with FAAH−/− mice. In wild-type mice, robust PF7845-yne labeling was observed throughout the brain that showed high overlap with FAAH immunostaining and mRNA HCR (Choi et al., 2018; Sylwestrak et al., Cell 164, 792-804, 2016). In contrast, negligible PF7845-yne signals were detected in FAAH−/− mice. Taken together, pharmacological and genetic results demonstrated that CATCH could visualize target engagement with high specificity in tissues from drug-treated mice. In addition, the inventors demonstrated with multiple rounds of click reaction, drug binding can be visualized in large tissue volume for 3D characterizations.

### Example 2. CATCH Reveals Brain-Wide Drug-Target Interactions and Detects Rare Targets

As exemplification, CATCH reveals brain-wide drug-target interactions and detects rare targets. Having verified the ability of CATCH to detect interactions between PF7845-yne and FAAH, the inventors evaluated its broader utility by investigating two other covalent drug-target combinations: the structurally distinct FAAH inhibitor BIA-10-2474 (Kiss et al., 2018) and the monoamine oxidase (MAO) inhibitor pargyline (Krysiak et al., 2012). Like PF7845, BIA10-2474 has a urea backbone and inhibits FAAH via a nucleophilic substitution reaction with its active serine residue (Kiss et al., 2018). In contrast, pargyline inhibits MAO by forming a covalent adduct via an oxidation-addition reaction (Krysiak et al., 2012). Alkyne analogs of both drugs have been developed in previous studies (Huang et al., 2019; Krysiak et al., 2012). CATCH imaging revealed similar labeling patterns following in vivo administration of PF7845-yne and BIA10-2474-yne, characterized by strong signals in the neocortex, thalamus and hippocampus. In contrast, pargyline-yne administration resulted in a distinct pattern of labeling that was enriched in hypothalamus, pons, and lateral ventricles. CATCH permitted immunostaining of FAAH after CuAAC reaction, confirming that PF7845-yne and BIA10-2474-yne signals are predominantly associated with FAAH (R²=0.95 and 0.84, respectively, simple linear regression, same for the following R² values). No such correlation was observed in pargyline-yne-treated animals (R²=0.03). On the contrary, MAO-A staining revealed no correlation for PF7845-yne (R²=0.005), but better correlation for pargyline-yne (R²=0.43). Imaging at higher resolution revealed that the FAAH inhibitors primarily targeted neuron-like structures in the neocortex and hippocampus. Conversely, pargyline-yne was predominantly bound to vasculature-like structures throughout the brain, with the exception of sparse but specific labeling of neuron-like structures in the hypothalamus and pons.

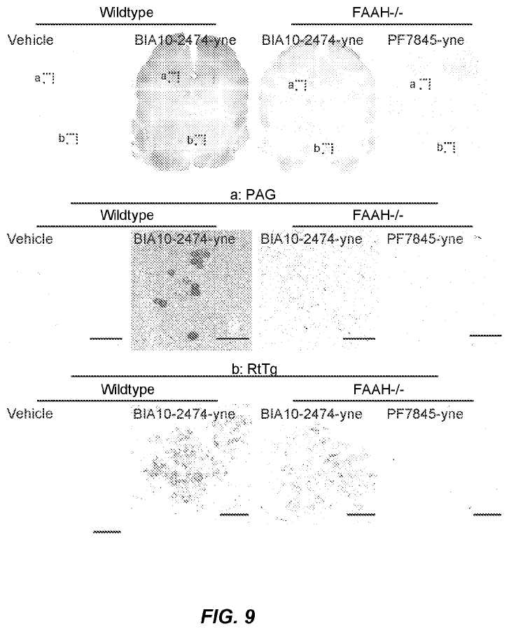

Unanticipated toxicity of BIA-10-2474 has been reported in humans, likely due to greater off-target activity compared to PF7845 (Kerbrat et al., 2016; Van Esbroeck et al., 2017). Accordingly, the inventors observed a lower correlation between FAAH expression and BIA-10-2474-yne CATCH labeling than PF7845-yne (R²=0.84 vs 0.95). Furthermore, although the majority of PF7845-yne and BIA-10-2474-yne CATCH signal was abolished in FAAH−/−mice, including in the S1, CA1, and periaqueductal gray (PAG), surprisingly, some BIA-10-2474-yne labeling remained in the reticulo tegmental nucleus of the pons (RtTg) (FIG. 9). These data indicated that BIA10-2474-yne binds to off-target sites in the RtTg and also demonstrated the ability of CATCH to uncover drug targets at cellular resolution.

### Example 3. Using CATCH and Fluorescent Labeling to Identify Target Cell Types

As additional exemplification of the present invention, it was found that CATCH can be multiplexed with fluorescent labeling to identify target cell types. Understanding the cell types affected by drug treatment in vivo is critical for accurately interpreting pharmacological mechanism of action, particularly for drugs targeting the CNS, which harbors diverse neural substrates and cell types. Having established that CATCH can visualize drug targets with single-cell resolution, the inventors investigated whether it could be combined with molecular markers of cell identity to characterize and register cellular target identities. The inventors demonstrated PF7845-yne engages neurons, but not blood vessels in S1, as revealed by NeuN and lectin staining (FIG. 5). The inventors performed similar antibody and mRNA staining in a wide range of brain regions of interest, including but not limited to cortex, hippocampus, hypothalamus, striatum, thalamus, basal lateral amygdala, pons, cerebellum, and brainstem and characterized PF7845, BIA-10-2474 and pargyline binding cell types. In addition to neuron and blood vessel staining, the inventors have performed long range projecting neuron identifications via Ctip2 antibody staining; Parvalbumin (PV) interneurons via PV antibody staining; somatostatin (SST) interneurons via SST mRNA hybridization chain reaction (HCR); astrocyte via glial fibrillary acidic protein (GFAP) antibody staining; FAAH expression via FAAH antibody staining and FAAH mRNA HCR; MAO-A expression via MAO-A antibody staining; noradrenergic (NA) neurons via tyrosine hydroxylase (TH) antibody staining, inhibitory neurons via VGAT-Ai14 transgenic mice; neuronal soma via MAP2 antibody staining; neuronal pre-synaptic terminals via synapsin antibody staining.

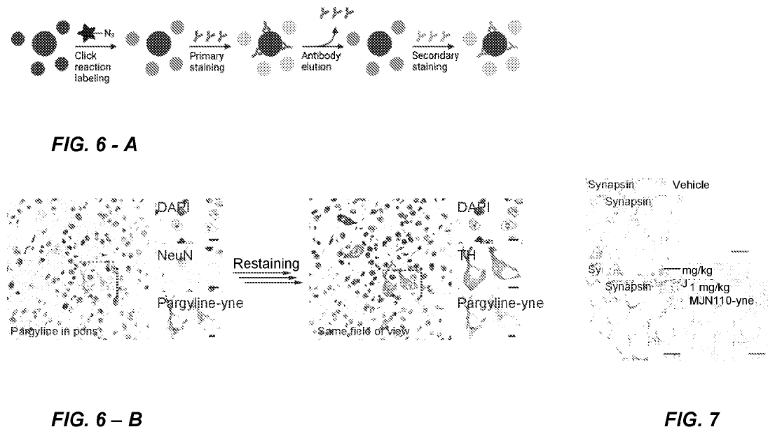

With the stable polyacrylamide hydrogel network, the inventors tested the possibility for reversible, multiplexed marker staining (FIG. 6, A). The inventors identified a small group of neuron-like structures in the pons that were NeuN negative but targeted by pargyline-yne (FIG. 6, B). Based on MAO gene expression data from the Allen Brain Atlas, the inventors wondered if these cells represented NA neurons in the locus coeruleus (LC) of the pons, which are commonly identified by TH staining (Schmidt et al., 2019). The inventors tested this by stripping off NeuN immunostaining from the brain sections and re-staining staining with a TH antibody on the same sample. CATCH signals resulting from the CuAAC conjugation of Alexa647 were resistant to the high temperature and SDS elution protocol used to remove NeuN staining and revealed colocalization of pargyline-yne and TH in the same cells (FIG. 6, B).

### Example 4. CATCH can Reveal Drug Binding at Subcellular Resolution

In another exemplification, the claimed invention was able to reveal drug binding at subcellular resolution. Specifically, the inventors asked whether CATCH could image drug-target interactions in subcellular compartments in tissue. To this end, the inventors targeted monoacylglycerol lipase (MAGL), a key enzyme involved in terminating the signaling function of endocannabinoid 2-archidonoylglycerol (2-AG) (Di Marzo, 2018), using a well characterized alkyne analogue of the MAGL inhibitor MJN110 (Chang et al., ACS Chem Biol 8, 1590-1599, 2013; Niphakis et al., ACS Chem Neurosci 4, 1322-1332, 2013). The inventors confirmed the specificity of MJN110-yne signals generated by CATCH using both pharmacological pretreatment of the parent compound MJN110 and MAGL−/−mice.

As previously reported, the inventors found that MJN110-yne selectively engaged MAGL at 1 mg/kg and showed substantial off-target engagement of FAAH at 20 mg/kg in gel-based CC-ABPP of whole brain lysates. Because MAGL is restricted in localization to presynaptic axonal terminals, whereas FAAH is expressed in neuronal soma, the inventors sought to determine whether CATCH could detect on-and off-target MJN110-yne binding across subcellular compartments. The inventors focused on the well-characterized CA3 to CA1 projection in the hippocampus as it allowed us to observe both axon terminals and soma in the same field of view. As expected, the inventors observed an overall higher fluorescence intensity in the hippocampus treated with 20 mg/kg MJN110-yne. Higher resolution imaging combined with synapsin and microtubule-associated protein 2 (MAP2) immunostaining revealed that MJN110-yne signal was restricted to synapsin positive axon terminals at a low dose (1 mg/kg, FIG. 7), and spread to the neuronal soma at the high dose (20 mg/kg). Pre-treating animals with PF7845 significantly suppressed MJN110-yne binding in the soma, but not axon terminals, indicating that these signals originated from the off-target FAAH binding and on-target MAGL binding, respectively. These data thus demonstrate that CATCH can visualize drug binding to different protein targets at subcellular resolution.

### Example 5. CATCH can Measure Dose-Dependent Drug-Target Engagement

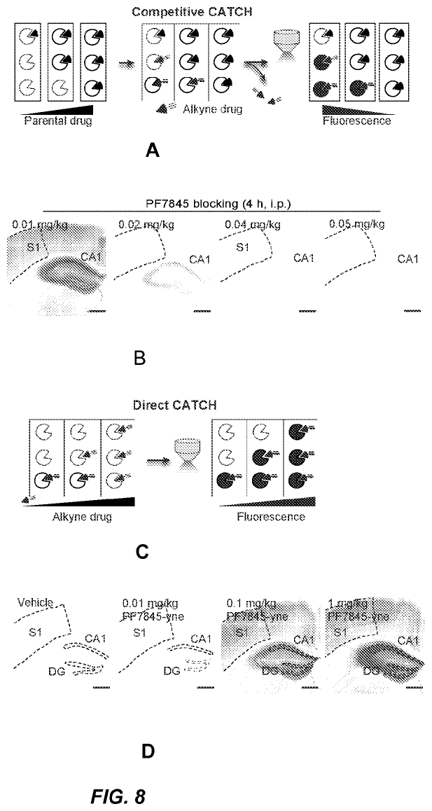

As a further exemplification, the inventors found that the claimed methods can be used to measure dose-dependent drug-target engagement. After establishing CATCH's specificity, scalability, compatibility, and resolution, finally, the inventors tested if CATCH can be used to quantitatively resolve dose-dependent target engagement after in vivo drug administration. Recognizing that the alkyne modifications made to drugs to create CATCH probes have the potential to alter pharmacokinetic (PK) properties, an outcome that the inventors confirmed for both the FAAH and MAGL alkyne probes, which shared similar blood brain barrier (BBB) permeability, and brain distribution to parent inhibitors, but showed altered in vivo half-lives, the inventors adopted a competitive strategy to visualize dose-dependency of drug-target interactions in vivo. In this competitive CATCH approach, animals are first treated with a dose range of the parental drugs, followed by a single high dose of the alkyne probes. The dose-dependent target engagement by the parental drug can be recorded based on the competitive blockade of signal in an anatomically specific manner (FIG. 8, A).

The inventors treated mice with 0.01-1 mg/kg of parental PF7845 followed by 1 mg/kg PF7845-yne probe to readout the dose-dependent brain targets of PF7845 by competitive CATCH. Consistent with previous in-gel analysis (Niphakis et al., ACS Chem Neurosci 3, 418-426, 2012), no target binding was detected at 0.01 mg/kg by CATCH whereas full FAAH inhibition was achieved at 0.05 mg/kg. Interestingly, the inventors found that, at an intermediary dose (0.02 mg/kg), FAAH was fully inhibited in the cortex but not in the hippocampus (FIG. 8, B). Specifically, a non-homogeneous, stripe-like pattern was observed in the hippocampal CA1 pyramidal cell layer and DG granular cell layer, suggesting sub-maximal drug engagement in these regions. Next, the inventors stained brain sections with lectin to mark vasculature structures, which revealed that regions with low probe intensity (i.e., cells pre-blocked by the parental PF7845) were associated with trespassing blood vessels. Conversely, the inventors adopted a direct approach to visualize dose-dependent alkyne drug PK properties (FIG. 8, C). Interestingly, when an intermediary dose of alkyne PF7845-yne (0.1 mg/kg; FIG. 8, D) was directly visualized by CATCH in the hippocampus, the inventors observed a mirrored intensity pattern where higher probe intensity was in proximity with the vasculature.

Together, both competitive and direct labeling suggested that PF7845-FAAH interactions at intermediary doses might be limited by the distance between FAAH-expressing neurons and surrounding capillaries. This would effectively create a drug gradient that results in sub-maximal engagement in neurons more distal to the vasculature. This heterogeneous distribution is unique to the hippocampus, suggesting that it might be attributed to particularly high levels of FAAH expression or relatively low vasculature volume in this region.

Finally, as covalent inhibitors typically process prolonged target engagement, the inventors found sustained FAAH blockage to competitive probe binding even 24 hours after PF7845 administration. Collectively, by complementing competitive and direct labeling strategies, CATCH can reveal dose-dependent, quantitative target engagement across heterogeneous brain regions that are not easily accessible by traditional lysate-based methods.

### Example 6. Exemplified Materials and Methods

Detailed information of some protocols and materials that can be employed in the practice of the present invention are set forth below.

Mouse model: Mice were group-housed on a 12-hr light dark cycle and fed a standard rodent chow diet. Both male and female, 6-9-week-old WT C57BL6J, FAAH−/−, MAGL−/− and VGAT-Ai14 mice were used. VGAT-Ai14 mice were obtained by crossing VGAT-ires-cre mice with Ai14 mice. All experimental protocols were approved by the Scripps Research Institute Institutional Animal Care and Use Committee and were in accordance with the guidelines from the NIH.

Sample collection: Inhibitors were administered to mice in a vehicle of 10% DMSO, 2% Tween-80 in saline for intraperitoneal (i.p.) injections. After drug injection at designated dose and time, mice were heavily anesthetized with isoflurane and then transcardially perfused with ice cold PBS followed by ice cold 4% PFA in PBS with sucrose. Mouse brains or other peripheral tissues were then dissected out and post fixed in 4% PFA overnight at 4° C. Tissues were washed with PBS, embedded in 2% agarose and sectioned as 100-micron or 500-micron tissue sections by vibratome (Leica VT1000S). Tissue sections were stored in PBS with 0.02% sodium azide at 4° C. for further processing.

For in gel ABPP analysis, after anesthetizing, mice were decapitated and target tissues were harvested and flash frozen in liquid N₂ without perfusion or fixation. Each mouse hemisphere and liver were washed with ice cold PBS on ice (2×1 mL) to remove excessive blood. Tissues were homogenized in 1 mL PBS and sonicated for 10 min in ice cold water. Tissues were centrifuged (1000 g, 10 min, 4° C.) and supernatant was then centrifuged at high speed (100,000 g, 45 min, 4° C.). Supernatant was discarded and remaining pellets were gently washed with ice cold PBS (2×0.5 mL). Pellets were resuspended by gentle pipetting and protein concentrations were quantified by the Bio-Rad DC Protein Assay Kit. Proteomes were then diluted to 1.0 mg/kg for immediate use or aliquoted and stored at −80° C.

For quantitative PK studies, at each collection timepoint (1 h, 2 h, 4 h, and 24 h), blood was collected by cardiac puncture into EDTA microtubes and mixed. Exactly 100 μL of blood was added to 400 μL of ice-cold acetonitrile to immediately inactivate blood esterase activity. Mice were then perfused using ice-cold PBS for 2 minutes at 5 mL/min until perfusate ran clear. The brain was collected in four sections (forebrain, cerebellum, brainstem, and backup forebrain) and snap frozen in liquid nitrogen. All acetonitrile-extracted blood samples were centrifuged at 17,000×G for 3 minutes and the supernatant was transferred to new tube and frozen at −80° C. until bioanalysis.

In gel click chemistry ABPP assay: Click chemistry labeling of alkyne inhibitors was performed as previously reported (Niphakis et al., ACS Chem Neurosci 3, 418-426, 2012). Click reaction buffer was prepared by mixing CuSO₄ (50 mM in H₂O, 1.0 μL/reaction), TBTA (1.7 mM in 1:4 DMSO/t-BuOH, 3.0 μL/reaction), freshly prepared TCEP (50 mM in H₂O, 1.0 μL/reaction) and 5-TAMRA azide (1.25 mM in DMSO, 1.0 μL/reaction). For every 50 μL tissue proteome (1.0 mg/kg), 6 μL click buffer was added. Reaction mixture was gently mixed and kept in a dark drawer for 1 hour at RT. Reactions were quenched by addition of SDS loading buffer (4×, 18 μL) and run on SDS-PAGE.

### Tissue Clearing

CLARITY: PFA fixed tissues were incubated in A1P4 hydrogel (1% acrylamide, 0.125% Bis, 4% PFA, 0.025% VA-044 initiator (w/v), in 1× PBS at 4° C. for CLARITY embedding as previously published (Sylwestrak et al., Cell 164, 792-804, 2016). Samples were kept overnight with gentle agitation to allow sufficient monomer diffusion. Samples were flushed with nitrogen and degassed for 15 min at RT. After degassing, samples were polymerized at 37° C. for 4 hours with gentle agitation. Samples were removed from hydrogel and washed with 8% PBS-SDS (pH=7.0) at 40° C. for two days. After clearing, samples were washed with PBST (pH=7.0 with 0.2% Triton-X100, same for the following) 3×10 min at RT to remove residue SDS. Samples were briefly washed with PBS and then stored in PBS with 0.02% sodium azide at 4° C.

SHIELD: SHIELD processing was carried out as previously reported (Park et al., Nat. Biotechnol. 37, 73-83, 2019). Tissue samples were incubated in SHIELD-OFF solution (25% dH₂O, 25% SHIELD BUFFER solution, 50% SHIELD epoxy solution) at 4° C. for 1 day with gentle agitation. Samples were then transferred to an equivalent volume of SHIELD-ON buffer and incubated at 37° C. with shaking for 24 hours. Samples were washed with PBS 3×10 min at RT to remove residual SHIELD-ON buffer and then cleared with 8% PBS-SDS (pH=7.0) overnight at 40° C. Samples were washed with PBST 3×10 min at RT to remove residue SDS. Samples were briefly washed with PBS and then stored in PBS with 0.02% sodium azide at 4° C.

iDISCO: iDISCO was adapted from published protocols (Renier et al., Cell 159, 896-910, 2014). All the washes with organic solvents were carried out at 4° C. with shaking. Fixed samples were washed in 20%, 40%, 60%, 80% methanol in H₂O/0.1% Triton X-100/0.3 M glycine (B1N buffer, pH 7), and then with 100% methanol twice. Samples were then delipidated with 100% dichloromethane (DCM), washed in 100% methanol three times, then in 80%, 60%, 40%, 20% methanol in BIN buffer. Each round of wash above was 15 min. Samples were then washed with PBST 3×10 min at RT to remove residue organic solvent. Samples were briefly washed with PBS and then stored in PBS with 0.02% sodium azide at 4° C.

fDISCO: fDISCO was adapted from published protocols (Qi et al., Sci. Adv. 5, eaau8355, 2019). Samples were washed at RT with shaking. Fixed samples were washed in 50%, 70%, 80% tetrahydrofuran (THF) in 25% Quadrol (in 1× PBS to adjust to pH 9), then 100% THF twice. Samples were delipidated with 100% DCM, then washed with 100% THF three times followed by 80%, 70%, 50% THF. Each round of wash above was 15 min. Samples were then washed with PBST 3×10 min at RT to remove residual organic solvent. Samples were briefly washed with PBS and then stored in PBS with 0.02% sodium azide at 4° C.

CUBIC 3.0: CUBIC protocol was adapted from the recent CUBIC3.0 protocol (Tainaka et al., Cell Rep 24, 2196-2210, 2018). Samples were cleared with CUBIC-L buffer (10% wt N-butyldiethanolamine, 10% wt TritonX-100 in dH₂O) at 37° C. for 4 hours. Samples were then washed with PBST 3×10 min at RT to remove residual detergents. Samples were briefly washed with PBS and then stored in PBS with 0.02% sodium azide at 4° C.

CATCH labeling: For 100-micron sections labeling, the full CATCH reaction buffer contains: 5 μM AF647-picolyl azide, 150 μM CuSO₄, 300 μM BTTP, 2.5 mM sodium ascorbate, and 10% DMSO in PBS. Tissue sections were incubated in click incubation buffer (without sodium ascorbate) overnight at RT. Tissues were then transferred to newly prepared incubation buffer and 100 mM freshly prepared sodium ascorbate was subsequently added to initiate the reaction. After 1 hour at RT in the dark with minor agitation, the reaction was quenched by addition of 4 mM EDTA in PBS (pH=8.0) and samples were washed 3×10 min with PBST. After removing click reaction cocktail, samples were stained by DAPI (1:3000 dilution in PBS from 10 μM stock) for 15 min at RT and would be ready for RI matching and imaging. They could also undergo further staining for cell type registration. For 500-micron sections, 300 μM CuSO₄ and 600 μM BTTP were used for incubation and reaction. To achieve full labeling, tissues were incubated at 37° C. for 2 days, and underwent 4 rounds of 1 h click reaction at RT.

Immunostaining: Samples were incubated with primary antibodies in PBST overnight at 4° C. unless otherwise noted. Samples were then washed with PBST, 3×30 min, RT and transferred to secondary antibodies diluted in PBST. Samples were incubated with secondary antibodies overnight, RT and washed with PBST 3×30 min. For antibody elution, imaged slides were dismounted, and tissues were washed by 8% PBS-SDS (pH=7.0) at 60° C. for 8 hours. Samples were then washed by PBST 3×10 min at RT to remove residual SDS and could undergo the next round of staining.

Hybridization Chain Reaction (HCR): Samples were pre-incubated in probe hybridization buffer for 30 min at 37° C. Samples were then transferred to new probe hybridization buffer with 16 nM FAAH-B1 or 4 nM SST-BI probe and incubated overnight at 37° C. Samples were washed by probe washing buffer at 37° C., 3×30 min and then by 5×SSCT (750 mM NaCl, 75 mM sodium citrate, 0.1% Tween-20 in H₂O), 2×30 min, RT. Samples were pre incubated in amplification buffer for 30 min at RT. HCR hairpin was typically stored as 3 μM, 12 μL aliquots. For every hairpin aliquot, 4 μL of 20×SSC (VWR, 10128-690) was added. Hairpins were heated to 95° C. for 90 seconds and cooled to RT in a dark drawer. Hairpins were then added to new amplification buffer to afford final hairpin concentration of 360 nM. Samples were then transferred to hairpin containing amplification buffer and incubated overnight at RT. Samples were washed with 5×SSCT, 3×30 min and would be ready for imaging.

Confocal microscopy: Labeled samples were immersed in RI matching media (RapiClear, 1.45 RI), then mounted to a glass microscope slide. Tissues were then imaged with the Olympus FV3000 confocal microscope with a 10×, 0.6 NA, water immersion objective (XLUMPlanFI, Olympus) for global characterizations, or a 40×, 1.25 NA, silicone oil immersion (UPlanSApo, Olympus) for detailed cell type, mRNA expression and subcellular compartment identification.

Drug in vivo concentration analysis: Compound concentration in blood and brain regions (forebrain and brainstem) was determined by LC/MS/MS. Acetonitrile-extracted blood samples were prepared as described above and the blood calibration curves were made using blood from naïve mice quenched following the same protocol spiked with serial dilutions of the test article, ranging 0.1-5000 ng/mL.

To each frozen brain tissue, 3 volumes (in μL) of ice-cold acetonitrile: water 3:1 (vol:vol) was added by tissue weight (mg) for compound extraction. Tissues were then homogenized for one minute at 30 Hz in a tissue lyser (Tissue Lyser II, Qiagen) with one stainless steel bead (5 mm diameter). Samples were incubated on ice for 45 minutes. Afterwards, samples were centrifuged at 2.400×g at 4 C for 15 minutes and supernatants were transferred to another tube. The forebrain and brainstem calibration curves were made using a brain homogenate from naïve mice (extracted following the same protocol than the samples) spiked with serial dilutions of the test article, ranging 0.1-5000 ng/mL.

10 μL of the extracts were injected onto an Agilent 1290 UPLC system equipped with a G7120A pumps, a G7129B autosampler and a G1170A column manager (Agilent Technologies). Chromatographic separation was achieved using an Acquity UPLC BEH C18 Column (2.1×50 mm, 1.7 μm particle size, 130 Å) coupled to an Acquity UPLC BEH C18 Column Guard (2.1×5 mm, 1.7 μm particle size, 130 Å) (Waters Corporation). Mobile Phase A was composed by Water/Acetonitrile 95:5 by vol. and mobile phase B was composed by Acetonitrile/Water 95:5 by vol. 0.1% formic acid was added to both mobile phases. Gradient started with 0% B that was kept for 0.5 min before being increased linearly to 100% B in 4 min. Afterwards, solvent B was kept at 100% for 1 min, before switching to the initial conditions in 0.1 min. System was allowed to equilibrate for 1.4 minutes before next sample injection. Flow rate was kept at 0.6 mL/min. Column was heated at 50° C.

Analytes were quantified using a 6460 triple quadrupole mass spectrometer equipped with an electrospray Jet Stream source (Agilent Technologies) operated in dynamic multiple reaction monitoring (dMRM) mode. The quantitative and qualitative transitions for each compound were optimized using the authentic standards in the Optimizer software (Agilent Technologies): MJN110 (Quant: 462.1→235, CE=10; Qual: 462.1→165, CE=45), MJN110-yne (Quant: 452.1→225, CE=10; Qual: 452.1→189.6, CE=45), PF7845 (Quant: 456.2→335.1, CE=22; Qual: 456.2→122, CE=30) and PF7845-yne (Quant: 470.2→349.2, CE=18; Qual: 470.2→320.2, CE=30). The following parameters were kept constant for all transitions: Fragmentor=140, Cell Accelerator Voltage=4, Polarity-Positive. Total cycle time was 500 ms. Source parameters were kept as follows: Dry Gas Temperature=350° C., Dry Gas Flow=11 L/min, Sheath Gas Temperature=350° C., Sheath Gas Flow=11 L/min, Nebulizer=50 psi, Noozle voltage=1500 V (positive) and Capillary=3500 V (positive).

Compound concentrations were calculated by extrapolating the integrated area under the curve with the calibration curves for each particular compound prepared in the same matrix than the samples using the Masshunter Quantitative Analysis Software (Agilent Technologies).

Quantification and statistical analysis: Images were typically acquired with 10 um intervals and analyzed with Fiji-ImageJ for 2D quantifications. 3D drug binding was visualized by Imaris 9.2.1. All images were stored and processed as TIFF format.

Signal profile analysis: A 40-micron straight line was drawn across a pyramidal cell in cortex layer V on tissue surface as previously reported (Pan et al., Nat. Methods 13, 859-867, 2016). Signal profile was plotted along the line and data was recorded that included background, drug positive membrane pixels and nucleus. The plotted data was then normalized by dividing each value by the average intensity of all intensity values on the same line.

Click labeling signal analysis: Individual drug positive cell intensity was quantified as previously reported (Pan et al., Nat. Methods 13, 859-867, 2016; Qi et al., Sci. Adv. 5, eaau8355, 2019). A 150×150 pixel (pixel size 0.414 μm) ROI was generated on tissue surface in cortex layer V and an auto threshold was applied to measure the mean drug positive pixel intensity as I_{signal}. The mean intensity of the remaining pixel is used as background intensity as I_{background}. The mean labeling intensity is calculated as I _{labeling}=I_{signal}−I_{background}. The signal to background ratio as a function of tissue depth was calculated as the ratio of I_{signal} and I_{background} as previously reported (Sylwestrak et al., Cell 164, 792-804, 2016).

Regional drug abundance analysis: Images were stacked with max z projection covering the whole coronal brain section. Different ROIs were drawn to outline individual brain regions based on a mouse brain atlas (The Mouse Brain in Stereotaxic Coordinates, Second Edition) and their average intensity were recorded. Intensity values were then normalized to the average intensity of vehicle samples in the respective brain region.

Fluorescence intensity correlation analysis: To quantify the correlation of FAAH and MAO-A immunostaining signal and drug signal, images were first stacked by max intensity projection (MIP) to obtain a single plane image. The image was then compressed 10 times to obtain final pixel size of 24.9 μm. The whole tissue was then outlined by applying a threshold in the immunostaining channel and each X-Y coordinate intensity was saved in both channels. Intensity values for each pixel were then normalized to the average intensity of each measurement and plotted. Single linear regression was applied to analyze pixel wise signal correlation.

Drug binding capillary analysis: To quantify drug binding abundance in relation to the proximity to the nearest blood vessel, the pyramidal cell layer in CA1 and granular cell layer in DG were first cropped out. A threshold was applied to quantify the average nucleus (drug negative) pixels intensity as background intensity. Then different ROIs were cropped out based on distance to the nearest blood vessel and the same threshold value was applied to quantify average intensity in non-nucleus pixels. Average labeling intensity was then obtained by subtracting the average non-nucleus pixel intensity by the background intensity.

Subcellular compartment intensity analysis: For MJN110-yne characterization, images were acquired at 5 μm from the tissue surface where the highest immunostaining signal can be observed. Different cellular compartments were identified by applying a threshold in immunostaining signal and mean drug labeling intensity was measured in the selected threshold region. Three random squares were drawn in nucleus in different cells and their average intensity was used as background. The recorded intensity value in soma or axonal terminal was then subtracted by the intensity in the nucleus for background adjustment. Data was then normalized to the average intensity of the control group as indicated in each figure legends.

Unless otherwise specified, statistical analysis was evaluated with Graphpad Prism 9 using ordinary one-way and two-way ANOVA (* p<0.05; ** p<0.01; *** p<0.001; **** p<0.0001), followed by Tukey's multiple comparisons test, Šidák's multiple comparisons test or Dunnett's multiple comparisons test. All data presented as mean±SD with N given in figure legend as biological replicates unless otherwise noted. Brain wide correlation of immunostaining and drug intensity was evaluated with simple linear regression model and R squared was calculated for each drug. For intensity ratios across different subcellular compartments, unpaired Mann-Whitney (Wilcoxon rank-sum) test was used. Drug PK analysis was performed with t test.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

We claim:

1. A method for analyzing an drug molecule or metabolite thereof in a mammalian tissue, comprising (a) obtaining a tissue sample from a mammalian subject who has been administered the drug molecule, (b) clearing the tissue sample with a delipidation agent, (c) performing a CuAAC reaction with the cleared tissue sample to label the drug molecule, and (d) examining the drug molecule in the tissue sample with an analytical means.

2. The method of claim 1, wherein the drug molecule is a small molecule.

3. The method of claim 1, wherein the drug molecule is a covalent drug.

4. The method of claim 1, wherein the delipidation agent is a hydrogel based tissue clearing agent, a hydrophobic tissue clearing agent, or a hydrophilic tissue clearing agent.

5. The method of claim 1, wherein the drug molecule is labeled with a fluorophore via the CuAAC reaction.

6. The method of claim 1, wherein the CuAAC reaction is optimized.

7. The method of claim 6, wherein the optimized CuAAC reaction comprises a Cu²⁺ concentration used in the reaction that is from about 100 μM to about 150 μM.

8. The method of claim 6, wherein the CuAAC reaction is optimized by including a pre-incubation of the cleared tissue prior to initiation of the click reaction.

9. The method of claim 6, wherein the CuAAC reaction is optimized with a click reaction ligand that improves signal to noise ratio.

10. The method of claim 9, wherein the click reaction ligand is BTTP, THPTA or BTTAA.

11. The method of claim 1, wherein the drug molecule or metabolite thereof contains an alkyne group or analog thereof.

12. The method of claim 1, wherein the analytical means is an imaging means.

13. The method of claim 13, wherein the imaging means comprises confocal microscopy.

14. The method of claim 1, further comprising staining the tissue sample with an agent for a cell type that is known or suspected to express a target of the drug molecule.

15. The method of claim 14, wherein the agent is an antibody or nucleic acid probe that is specific for the cell type.

16. A method for identifying the target of a drug molecule in a mammalian tissue, comprising (a) obtaining a tissue sample from a mammalian subject who has been administered the drug molecule, (b) clearing the tissue sample with a delipidation agent, (c) performing a CuAAC reaction with the cleared tissue sample to label the drug molecule, and (d) examining the drug molecule in the tissue sample with an analytical means to identify the target of the drug molecule in the tissue; thereby identifying the target of the drug molecule.

17. The method of claim 16, wherein the drug molecule is a small molecule.

18. The method of claim 16, wherein the drug molecule is a covalent drug.

19. The method of claim 16, wherein the delipidation agent is a hydrogel based tissue clearing agent, a hydrophobic tissue clearing agent, or a hydrophilic tissue clearing agent.

20. The method of claim 16, wherein the CuAAC reaction is optimized.

21. The method of claim 16, wherein the binding target in the tissue sample is identified via immunostaining, RNA hybridization, or a spatially-resolved molecular characterization means.
